(19) United States
(12) Patent Application Publication
(10) Pub. No.: US 2015/0044718 A1
(43) Pub. Date: Feb. 12, 2015

(54) ON-COLUMN ENZYMATIC CLEAVAGE

(71) Applicant: Hoffmann-La Roche Inc., Little Falls, NJ (US)

(72) Inventors: Roberto Falkenstein, Muenchen (DE); Adelbert Grossmann, Eglfing (DE); Friederike Hesse, Muenchen (DE)

(21) Appl. No.: 14/381,130

(22) PCT Filed: Feb. 27, 2013
(86) PCT No.: PCT/EP2013/053872

§ 371 Date: Aug. 26, 2014

(30) Foreign Application Priority Data
Feb. 29, 2012 (EP) 12157510.4

(51) Int. Cl. C12P 21/06; C07K 14/775; C07K 14/65; C07K 14/47

(52) U.S. Cl. CPC C12P 21/06; C07K 14/4726; C07K 14/775; C07K 14/65
USPC 435/68.1

(57) ABSTRACT

Herein is reported a method for obtaining a polypeptide by an immobilized metal ion affinity chromatography from a pro-polypeptide that comprise at its N- or C-terminus an metal ion affinity chromatography tag and a protease cleavage site comprising the step of recovering the polypeptide from the immobilized metal ion affinity chromatography column by incubating the bound pro-polypeptide with a protease, whereby the immobilized metal ion affinity chromatography material has been washed at least once with an urea solution.

The current invention is in the field of polypeptide purification and polypeptide production using immobilized metal ion affinity chromatography combined with enzymatic removal of the metal ion affinity chromatography tag by on-column cleavage. Thus, herein is reported a method for the on-column cleavage of metal ion affinity chromatography tag containing polypeptides using proteases.

### BACKGROUND OF THE INVENTION

Proteins play an important role in today's medical portfolio. Expression systems for the production of recombinant polypeptides are well-known in the state of the art. Polypeptides for use in pharmaceutical applications are mainly produced in prokaryotic cells, such as E. coli, and mammalian cells such as CHO cells, NS0 cells, Sp2/0 cells, COS cells, HEK cells, BHK cells, PER.C6® cells, and the like.

For human application every pharmaceutical substance has to meet distinct criteria. To ensure the safety of biopharmaceutical agents to humans, for example, nucleic acids, viruses, and host cell proteins, which would cause severe harm, have to be removed. To meet the regulatory specification one or more purification steps have to follow the manufacturing process. Among other, purity, throughput, and yield play an important role in determining an appropriate purification process.

Different methods are well established and widespread used for protein purification, such as affinity chromatography with microbial proteins (e.g. protein A or protein G affinity chromatography), ion exchange chromatography (e.g. cation exchange (sulfopropyl or carboxymethyl resins), anion exchange (amino ethyl resins) and mixed-mode ion exchange, thiophilic adsorption (e.g. with thioether ligands), hydrophobic interaction or aromatic adsorption chromatography (e.g. with phenyl-sepharose, aza-arenophilic resins, or m-aminophenylboronic acid), metal ion chelate affinity chromatography (e.g. with Ni(II)- and Cu(II)-affinity material), size exclusion chromatography, and electrophoretical methods (such as gel electrophoresis, capillary electrophoresis) (see e.g. Vijayalakshmi, M. A., Appl. Biochem. Biotech. 75 (1998) 93-102).

### SUMMARY OF THE INVENTION

It has been found that a pro-polypeptide that comprises a metal ion affinity chromatography tag (affinity tag) and a protease cleavage site can be purified and enzymatically cleaved by using an immobilized metal ion affinity chromatography (IMAC) based method. More precisely it has been found that the pro-polypeptide bound to the IMAC column has to be contacted, e.g. washed, at least once with a urea containing solution in order to allow succeeding process steps to be performed, i.e. the cleaved polypeptide can be recovered in a concentration and with a purity that enables succeeding large scale downstream (e.g. purification) process steps to be performed.

One aspect as reported herein is a method for producing a polypeptide from a pro-polypeptide, whereby the pro-polypeptide comprises at its N- or C-terminus a metal ion affinity chromatography tag and a protease cleavage site located between the tag and the polypeptide, by an on-column enzymatic cleavage of the protease cleavage site on an immobilized metal ion affinity chromatography column comprising the following steps (in the following order):
denaturing the pro-polypeptide bound to the metal ion affinity chromatography material,
renaturing the pro-polypeptide bound to the metal ion affinity chromatography material, and
incubating the bound pro-polypeptide with a protease and thereby producing the polypeptide.

In one embodiment the denaturing is by contacting the bound pro-polypeptide with a solution comprising a denaturing agent. In one embodiment the renaturing the pro-polypeptide is by contacting the denatured pro-polypeptide with a solution free of denaturing agents.

One aspect as reported herein is a method for producing a polypeptide from a pro-polypeptide, whereby the pro-polypeptide comprises at its N- or C-terminus a metal ion affinity chromatography tag and a protease cleavage site located between the tag and the polypeptide, by an on-column enzymatic cleavage of the protease cleavage site on an immobilized metal ion affinity chromatography column comprising the following steps:
contacting the bound pro-polypeptide with a solution comprising a denaturing agent,
optionally contacting the bound pro-polypeptide with a solution comprising urea or a urea derivatives if the solution comprising a denaturing agent employed in the previous step was free of urea or a urea derivative or contacting the bound pro-polypeptide with a solution comprising urea or a urea derivatives if the solution comprising a denaturing agent employed in the previous step comprised a mixture of urea or a urea derivative and a denaturing agent,
recovering the polypeptide from the immobilized metal ion affinity chromatography column by incubating the bound pro-polypeptide with a protease and thereby producing a polypeptide.

In one embodiment the method comprises a washing step directly prior to the incubation with the protease with a solution free of denaturing agents.

In one embodiment the metal ion affinity chromatography tag is at the N-terminus of the polypeptide.

In one embodiment the polypeptide is renatured prior to the recovering step. In one embodiment the polypeptide is recovered in native form.

In one embodiment the polypeptide is recovered in denatured form.

In one embodiment the urea or urea derivative has a concentration of from about 0.5 M to about 8 M. In one embodiment the urea or urea derivative has a concentration of from about 2 M to 8 M. In one embodiment the urea or urea derivative has a concentration of about 4 M.

In one embodiment the denaturing agent is selected from guanidinium chloride, urea, thiourea, and tetramethylurea. In one embodiment the denaturing agent is guanidinium chloride.

In one embodiment the denaturing agent has a concentration of from about 0.5 M to about 6 M. In one embodiment the denaturing agent has a concentration of from about 1.5 M to about 3 M. In one embodiment the denaturing agent has a concentration of about 2 M.

In one embodiment the denaturing agent is guanidinium chloride and has a concentration of from about 0.5 M to about 6 M. In one embodiment the concentration is of from about 1.5 M to about 3 M. In one embodiment the concentration is about 2 M.

In one embodiment the pro-polypeptide is applied in native form or in denatured form to the immobilized metal ion affinity chromatography material.

In one embodiment the immobilized metal ion affinity chromatography material is an immobilized zinc affinity chromatography material.

In one embodiment the protease is selected from IgA protease, trypsin, or granzyme B. In one embodiment the protease is IgA protease.

In one embodiment the solution free of denaturing agents comprises about 0.01 M to about 2 M of a buffering agent. In one embodiment the solution free of denaturing agents comprises about 0.5 M to about 1.5 M of a buffering agent. In one embodiment the solution free of denaturing agents comprises about 1 M to about 1.2 M of a buffering agent.

In one embodiment the solution free of denaturing agents comprises about 0.5 M to about 1.5 M Tris at a pH value of about pH 8. In one embodiment the solution free of denaturing agents comprises about 1 M to about 1.2 M Tris at a pH value of about pH 8.

In one embodiment the polypeptide is a non-glycosylated polypeptide.

In one embodiment the polypeptide is human apolipoprotein A-I or a fusion polypeptide comprising apolipoprotein A-I. In one embodiment the polypeptide is a tetranectin-apolipoprotein A-I fusion polypeptide. In one embodiment the polypeptide is a tetranectin-apolipoprotein A-I fusion polypeptide that has an amino acid sequence selected from SEQ ID NO: 01 to SEQ ID NO: 03.

In one embodiment the polypeptide is human insulin-like growth factor 1 (IGF-1) or a fusion polypeptide comprising insulin-like growth factor 1 (IGF-1). In one embodiment the polypeptide is an insulin-like growth factor 1 (IGF-1) polypeptide that has the amino acid sequence SEQ ID NO: 21.

One aspect as reported herein is a method for producing a polypeptide comprising the following step:
purifying a polypeptide obtained from the cultivation medium of a cultivation of a prokaryotic or eukaryotic cell comprising a nucleic acid encoding the polypeptide with an immobilized metal ion affinity chromatography method as reported herein and thereby producing the polypeptide.

In one embodiment the method comprises one or more of the following steps:
cultivating a prokaryotic or eukaryotic cell comprising a nucleic acid encoding the polypeptide, and/or
recovering the polypeptide from the cells or/and the cultivation medium, and/or
if the polypeptide is recovered in the form of inclusion bodies solubilizing and/or re-folding the polypeptide, and/or
purifying the polypeptide with an immobilized metal ion affinity chromatography method as reported herein and thereby producing the polypeptide.

In one embodiment the prokaryotic cell is an E. coli cell, or a bacillus cell, or a yeast cell.

In one embodiment the eukaryotic cell is a CHO cell, or a BHK cell, or a HEK cell, or a NS0 cell, or a Sp2/0 cell.

### DETAILED DESCRIPTION OF THE INVENTION

Herein is reported an immobilized metal ion affinity chromatography method for obtaining a polypeptide from a pro-polypeptide with on-column cleavage of the pro-polypeptide by a protease, whereby the method comprises in one embodiment a first washing step under denaturing conditions, e.g. with a denaturing agent such as guanidinium chloride containing solution, and a second washing step with urea or a urea derivative containing solution. Especially, the polypeptide obtained by a method as reported herein can be recovered from the column in native form.

It has been found that the polypeptide can be recovered from the column in a form that can be processed further in additional chromatography steps as i) the polypeptide is obtained with a concentration of more than 1 mg/ml, and ii) the guanidinium chloride used for washing the pro-polypeptide bound to the IMAC is removed, if the bound pro-polypeptide is washed with a urea or a urea derivative containing solution prior to the enzymatic cleavage.

The terms “applying to” and grammatical equivalents thereof denote a partial step of a purification method in which a solution containing a substance of interest to be purified is brought in contact with a stationary phase. This denotes that a) the solution is added to a chromatographic device in which the stationary phase is located, or b) that a stationary phase is added to the solution comprising the substance of interest. In case a) the solution containing the substance of interest to be purified passes through the stationary phase allowing for an interaction between the stationary phase and the substances in solution. Depending on the conditions, such as e.g. pH, conductivity, salt concentration, temperature, and/or flow rate, some substances of the solution are bound to the stationary phase and, thus, are removed from the solution. Other substances remain in solution. The substances remaining in solution can be found in the flow-through. The “flow-through” denotes the solution obtained after the passage of the chromatographic device irrespective of its origin. It can either be the applied solution containing the substance of interest or the buffer, which is used to flush the column or which is used to cause the elution of one or more substances bound to the stationary phase. In one embodiment the chromatographic device is a column, or a cassette. The substance of interest can be recovered from the solution after the purification step by methods familiar to a person of skill in the art, such as e.g. precipitation, salting out, ultrafiltration, diafiltration, lyophilization, affinity chromatography, or solvent volume reduction to obtain the substance of interest in purified or even substantially homogeneous form. In case b) the stationary phase is added, e.g. as a solid, to the solution containing the substance of interest to be purified allowing for an interaction between the stationary phase and the substances in solution. After the interaction the stationary phase is removed, e.g. by filtration, and the substance of interest is either bound to the stationary phase and removed therewith from the solution or the substance of interest is not bound to the stationary phase and remains in the solution.

The terms “buffered” or “comprising a buffering agent” denote a solution in which changes of pH due to the addition or release of acidic or basic substances is leveled by a buffer substance. Any buffer substance or agent resulting in such an effect can be used. In one embodiment the buffer substance is selected from phosphoric acid or salts thereof, acetic acid or salts thereof, citric acid or salts thereof, morpholine, 2-(N-morpholino) ethanesulfonic acid or salts thereof, imidazole or salts thereof, histidine or salts thereof, glycine or salts thereof, or tris (hydroxymethyl) aminomethane (TRIS) or salts thereof. In one embodiment the buffer substance is selected from imidazole or salt thereof or histidine or salts thereof. Optionally the buffered solution may also comprise an additional inorganic salt. In one embodiment the inorganic salt is selected from sodium chloride, sodium sulphate, potassium chloride, potassium sulfate, sodium citrate, and potassium citrate.

A “polypeptide” is a polymer consisting of amino acids joined by peptide bonds, whether produced naturally or synthetically. Polypeptides of less than about 20 amino acid residues may be referred to as “peptides”, whereas molecules consisting of two or more polypeptides or comprising one polypeptide of more than 100 amino acid residues may be referred to as “proteins”. A polypeptide may also comprise non-amino acid components, such as carbohydrate groups, metal ions, or carboxylic acid esters. The non-amino acid components may be added by the cell, in which the polypeptide is expressed, and may vary with the type of cell. Polypeptides are defined herein in terms of their amino acid backbone structure or the nucleic acid encoding the same. Additions such as carbohydrate groups are generally not specified, but may be present nonetheless.

The term “bind-and-elute mode” denotes a way to perform a chromatography purification method. Herein a solution containing a polypeptide of interest to be purified is applied to a stationary phase, particularly a solid phase, whereby the polypeptide of interest interacts with the stationary phase and is retained thereon. Substances not of interest are removed with the flow-through or the supernatant, respectively. The polypeptide of interest is afterwards recovered from the stationary phase in a second step by applying an elution solution.

The term “on-column cleavage” denotes a way to perform a chromatography purification method. Therein the polypeptide to be purified is bound as a pro-polypeptide, which contains a tag with which the pro-polypeptide can be bound by a chromatography material, and which contains a protease cleavage site between the tag and the polypeptide, and is recovered from the chromatography material by incubation with a protease that recognizes the cleavage site and, thus, effects cleavage of the tag and thereby liberates the polypeptide. The polypeptide can be found in solution whereas the tag remains bound to the chromatography material.

The term “inclusion body” denotes a dense intracellular mass of aggregated polypeptide of interest, which constitutes a significant portion of the total cell protein, including all cell components of a prokaryotic cell.

The term “refolded” or “renatured” refers to a polypeptide obtained from a denaturized form. Typically, the goal of refolding is to produce a protein having a higher level of activity than the protein would have if produced without a refolding step. A folded protein molecule is most stable in the conformation that has the least free energy. Most water soluble proteins fold in a way that most of the hydrophobic amino acids are in the interior part of the molecule, away from water. The weak bonds that hold a protein together can be disrupted by a number of treatments that cause a polypeptide to unfold, i.e. to denaturize. A folded protein is the product of several types of interactions between the amino acids themselves and their environment, including ionic bonds, Van der Waals interactions, hydrogen bonds, disulfide bonds and covalent bonds.

The terms “denatured” or “denaturized” as used herein refer to a polypeptide in which ionic and covalent bonds and Van der Waals interactions which exist in the molecule in its native or refolded state are disrupted. Denaturation of a polypeptide can be accomplished, for example, by treatment with 8 M urea or 6 M guanidinium chloride, reducing agents such as mercaptoethanol, heat, pH, temperature and other chemicals. Reagents such as 8 M urea or 6 M guanidinium chloride disrupt both the hydrogen bonds and the hydrophobic bonds, and if mercaptoethanol is also added, the disulfide bridges (S—S) which are formed between cysteines are reduced to two —S—H groups. Refolding of polypeptides which contain disulfide linkages in their native or refolded state may also involve the oxidation of the —S—H groups present on cysteine residues for the protein to reform the disulfide bonds. A “denaturized” polypeptide is a polypeptide wherein secondary, tertiary, and/or quaternary structures are not the native ones. The polypeptide in this non-native form may be soluble but concomitantly in a biologically inactive conformation. Or the polypeptide may be insoluble and in a biologically inactive conformation with e.g. mismatched or unformed disulfide bonds. This insoluble polypeptide can be, but need not be, contained in inclusion bodies.

The term “in denatured form” denotes the form of a polypeptide wherein it does not have a secondary, tertiary, and/or quaternary structure in which the polypeptide has its biological activity.

The term “in native form” denotes the form of a polypeptide wherein it has a secondary, tertiary, and/or quaternary structure in which the polypeptide has its biological activity. A “renatured” polypeptide is in its native form.

The term “free of denaturing agents” denotes that a denaturant or denaturing agent is not present in the applied (wash) solution. This means that a polypeptide contacted with a solution free of denaturing agents will exist in its native form.

The term “affinity chromatography” as used within this application denotes a chromatography method which employs an “affinity chromatography material”. In an affinity chromatography the polypeptides are separated based on their biological activity or chemical structure depending of the formation of electrostatic interactions, hydrophobic bonds, and/or hydrogen bond formation to the chromatographical functional group. To recover the specifically bound polypeptide from the affinity chromatography material either a competitor ligand is added or the chromatography conditions, such as pH value, polarity or ionic strength of the buffer are changed. An “affinity chromatography material” is a chromatography material which comprises a complex chromatographical functional group in which different single chromatographical functional groups are combined in order to bind only a certain type of polypeptide. This chromatography material specifically binds a certain type of polypeptide depending on the specificity of its chromatographical functional group. Exemplary “affinity chromatographical materials” are a “immobilized metal ion affinity chromatography material” such as Ni(II)-NTA (NTA=nitrilotriacetic acid), Zn(II)-IDA (IDA=iminodiacetic acid) or Cu(II)-NTA containing materials, for the binding of fusion polypeptides containing a hexahistidine tag or polypeptides with a multitude of surface exposed histidine, cysteine, and/or tryptophane residues, or an “antibody binding chromatography material” such a protein A, or an “enzyme binding chromatography material” such as chromatography materials comprising enzyme substrate analogues, enzyme cofactors, or enzyme inhibitors as chromatographical functional group, or a “lectin binding chromatography material” such as chromatography materials comprising polysaccharides, cell surface receptors, glycoproteins, or intact cells as chromatographical functional group. In one embodiment the affinity chromatography material is Zn(II)-IDA.

The term “immobilized metal ion affinity chromatography” as used within this application denotes a chromatography method which employs an “immobilized metal ion affinity chromatography material”. Metal ion affinity chromatography is based on the formation of chelates between a metal ion, such as Cu(II), Ni(II) or Zn(II), which is bound to a bulk material as chromatographical functional groups, and electron donor groups of surface exposed amino acid side chains of polypeptides, especially with imidazole containing side chains and thiol group containing side chains. The chelate is formed at pH values at which those side chains are at least partly not protonated. The bound polypeptide can be recovered from the chromatography material by a change in the pH value, i.e. by protonation.

Exemplary “immobilized metal ion affinity chromatography materials” are HiTrap Chelating HP (GE Healthcare Europe GmbH, Germany), or Fractogel EMD Chelate (Merck, Darmstadt, Germany).

Methods for purifying polypeptides are well established and widespread used. They are employed either alone or in combination. Such methods are, for example, affinity chromatography using thiol ligands with complexed metal ions (e.g. with Ni(II)- and Cu(II)-affinity material) or microbial-derived proteins (e.g. protein A or protein G affinity chromatography), ion exchange chromatography (e.g. cation exchange (carboxymethyl resins), anion exchange (amino ethyl resins) and mixed-mode exchange chromatography, thiophilic adsorption (e.g. with thioether ligands), hydrophobic interaction or aromatic adsorption chromatography (e.g. with phenyl-sepharose, aza-arenophilic resins, or m-aminophenylboronic acid), size exclusion chromatography, and preparative electrophoretic methods (such as gel electrophoresis, capillary electrophoresis).

The term “enzymatic cleavage site” denotes a sequence of amino acid residues connected to each other via peptides bonds that can specifically be cleaved by a protease. In one embodiment the protease cleavage site is an IgA-protease cleavage site, or a Granzyme B cleavage site, or a Tev protease cleavage site, or a Prescission protease cleavage site, or a Thrombin cleavage site, or a Factor Xa cleavage site, or a Trypsin cleavage site, or a Chymotrypsin cleavage site, or an Enterokinase cleavage site.

In one embodiment the protease is IgA-protease, Granzyme B, Tev protease, Prescission protease, Thrombin, Factor Xa, Trypsin, Chymotrypsin, or Enterokinase.

The term “IgA-protease” denotes a protease derived from Neisseria gonorrhoeae with a recognition site comprising one of the following sequences wherein “↓” denotes the position of the cleaved bond:

| (SEQ ID NO: 05) | |
|---|---|
| Pro-Ala-Pro ↓ Ser-Pro, | |
| | (SEQ ID NO: 06) |
| Pro-Pro ↓ Ser-Pro, | |
| | (SEQ ID NO: 07) |
| Pro-Pro ↓ Ala-Pro, | |
| | (SEQ ID NO: 08) |
| Pro-Pro ↓ Thr-Pro, | |
| | (SEQ ID NO: 09) |
| Pro-Pro ↓ Gly-Pro, | |
| | (SEQ ID NO: 10) |
| Pro-Arg-Pro-Pro ↓ Thr-Pro, | |
| | (SEQ ID NO: 11) |
| Val-Val-Ala-Pro-Pro ↓ Ala-Pro, | |
| | (SEQ ID NO: 12) |
| Val-Val-Ala-Pro-Pro ↓ Ser-Pro, | |
| | (SEQ ID NO: 13) |
| Val-Val-Ala-Pro-Pro ↓ Thr-Pro, | |
| | (SEQ ID NO: 14) |
| Val-Val-Ala-Pro-Pro ↓ Gly-Pro, | |
| | (SEQ ID NO: 15) |
| Ala-Pro-Pro-Ala ↓ Ala-Pro, | |
| | (SEQ ID NO: 16) |
| Pro-Arg-Pro-Pro ↓ Ala-Pro, | |
| | (SEQ ID NO: 17) |

Pro-Arg-Pro-Pro ↓ Ser-Pro,
(SEQ ID NO: 18)
Pro-Arg-Pro-Pro ↓ Gly-Pro

wherein the first three are more frequently chosen and cleaved.

It has been found that in a method as reported herein using the on-column cleavage of a pro-polypeptide, i.e. in which the liberation of a polypeptide from a pro-polypeptide is effected while the pro-polypeptide is bound to a chromatography column and in which the pro-polypeptide has been washed with a guanidinium chloride containing solution, the column has to be at least once washed with a urea or a urea derivative containing solution.

In one embodiment the column is washed prior to the incubation of the pro-polypeptide, which is bound to an immobilized metal ion affinity chromatography material, with a protease with a solution comprising urea or a urea derivative.

In one embodiment the column is washed after the incubation of the pro-polypeptide with a protease with a solution comprising urea or a urea derivative. In one embodiment by washing with the urea or urea derivative containing solution the polypeptide is recovered from the immobilized metal ion affinity chromatography column.

In one embodiment the wash with the urea or urea derivative containing solution is immediately succeeded, i.e. prior to the incubation with the protease, by a wash with a buffered solution (solution comprising a buffering agent) that is free of a denaturing agent.

During the wash with denaturing agents such as guanidinium salts or urea e.g. endotoxins, if the polypeptide was produced in E. coli, can be removed.

During a wash step with a denaturing agent the polypeptide is either denatured if it was present in native form prior to the wash step or it is maintained in denatured form if it was already present in denatured form.

It has been found that the removal of other denaturing agents beside urea or urea derivatives prior to the on-column cleavage of a pro-polypeptide with a protease improves the on-column cleavage of the pro-polypeptide and allows succeeding process steps to be performed.

During the wash with a buffered solution free of a denaturing agent, i.e. under native conditions, the pro-polypeptide is returned into its native state while remaining bound to the immobilized metal ion affinity chromatography material.

Therefore, one aspect as reported herein is a method for obtaining or purifying a polypeptide comprising the following step:
recovering the polypeptide from an immobilized metal ion affinity chromatography column by incubating a bound pro-polypeptide with a protease,
   whereby the pro-polypeptide bound to the immobilized metal ion affinity chromatography material has been washed at least once with an urea or urea derivative containing solution.

In one embodiment the wash with the urea containing solution is prior to the application of the protease or after the incubation with the protease. If the washing with the urea containing solution is after the incubation with the protease the washing step is at the same time the recovering step of the polypeptide.

The method as reported herein is, thus, operated in bind-and-elute mode, i.e. the pro-polypeptide is first bound to the immobilized metal ion affinity chromatography material and thereafter, in a further step, the polypeptide is recovered from the metal ion affinity chromatography material by cleavage of the protease cleavage site in the pro-polypeptide. Intermittent wash steps can be included in the methods as reported herein.

The terms “denaturant” or “denaturing agent” denotes compounds that transfer a polypeptide from its native form in a non-native, i.e. denatured, form. Denaturants are generally chaotropic agents. Exemplary denaturants are urea and urea-derivatives (e.g. thiourea and tetramethylurea), guanidine and guanidine-derivatives (e.g. guanidinium chloride), tetraalkyl ammonium salts, long chain sulfonic acid esters, and lithium perchlorate. The term “denaturing agent” may also be understood as a mixture of denaturing agents.

In one embodiment the denaturing agent is urea or a urea-derivative.

In one embodiment the denaturing agent is guanidinium chloride.

In one embodiment the denaturing agent is urea. In one embodiment the urea has a concentration of from 2 M to 8 M.

In one embodiment the denaturing agent is thiourea. In one embodiment the thiourea has a concentration of from 1.5 M to 3 M.

In one embodiment of the aspects as reported herein the method for purifying or producing a polypeptide comprises the following steps:
applying a solution comprising the pro-polypeptide to the immobilized metal ion affinity chromatography material and thereby binding the pro-polypeptide to the immobilized metal affinity chromatography material,
optionally washing the immobilized metal ion affinity chromatography material with a solution comprising guanidinium chloride and thereby removing unwanted polypeptides,
washing the immobilized metal ion affinity chromatography material with a solution comprising urea and thereby conditioning the pro-polypeptide for enzymatic cleavage and the polypeptide for further downstream processing, and
recovering the polypeptide from the immobilized metal ion affinity chromatography column by incubating the bound pro-polypeptide with a protease.

Polypeptides (including pro-polypeptides) can be produced recombinantly in eukaryotic and prokaryotic cells, such as CHO cells, HEK cells, and E. coli cells. If the polypeptide is produced in prokaryotic cells it is generally obtained in the form of insoluble inclusion bodies. The inclusion bodies can easily be recovered from the prokaryotic cell and the cultivation medium. The polypeptide obtained in insoluble form in the inclusion bodies has to be solubilized before purification and/or re-folding procedure can be carried out.

Thus, a second aspect as reported herein is a method for producing a polypeptide comprising the following steps:
cultivating a prokaryotic or eukaryotic cell comprising a nucleic acid encoding the polypeptide,
recovering the polypeptide from the prokaryotic or eukaryotic cells or/and the cultivation medium,
optionally if the polypeptide is recovered in form of inclusion bodies solubilizing and/or re-folding the polypeptide,
purifying the polypeptide with an immobilized metal ion affinity chromatography method as reported herein and thereby producing a polypeptide.

In one embodiment the immobilized metal ion affinity chromatography method comprises the following steps:
applying a solution comprising the pro-polypeptide to the immobilized metal ion affinity chromatography material, whereby the solution comprises a denaturing agent or is free of denaturing agents,
optionally washing the immobilized metal ion affinity chromatography material with a solution comprising guanidinium chloride,
washing the immobilized metal ion affinity chromatography material with a solution comprising urea or a urea derivative, and
recovering the polypeptide from the immobilized metal ion affinity chromatography column by incubating the bound pro-polypeptide with a protease.

In one embodiment the polypeptide is a pro-polypeptide.

In the following different embodiments of all aspects as reported before are presented.

In one embodiment the immobilized metal ion affinity chromatography material is in a first step conditioned with a buffered solution. This solution can but needs not to comprise a denaturant. The buffering agent of the conditioning solution can be the same or different from the buffering agent of the solution comprising the pro-polypeptide.

Thereafter a solution comprising the pro-polypeptide is applied to the conditioned immobilized metal ion affinity chromatography material. In this step the pro-polypeptide is retained on (bound to/adsorbed to) the immobilized metal ion affinity chromatography material. This solution can but needs not to comprise a denaturant. The buffering agent of the loading solution can be the same or different from the buffering agent of the following wash solution.

Optionally after the loading of the immobilized metal ion affinity chromatography material with the pro-polypeptide a washing solution can be applied to the loaded immobilized metal ion affinity chromatography material. This solution comprises as (sole) denaturant guanidinium chloride.

To the immobilized metal ion affinity chromatography material with the bound pro-polypeptide a washing solution is applied. This solution comprises as sole denaturant urea or a urea derivative.

Optionally after the washing step(s) the pro-polypeptide is while being still bound to the immobilized metal ion affinity chromatography material transferred into a native form by washing the immobilized metal ion affinity chromatography material with bound pro-polypeptide with a buffered solution free of denaturing agents.

Finally for recovering the polypeptide from the immobilized metal ion affinity chromatography material the column is incubated with a protease that specifically cleaves the protease cleavage site between the immobilized metal ion chromatography affinity tag and the polypeptide. After the incubation the cleaved pro-polypeptide, i.e. the polypeptide, is recovered from the flow-through of the column. The recovering can optionally be effected by a denaturing agent containing solution.

In one embodiment the method for purifying or obtaining a polypeptide is a column chromatography method.

The volume of the different solutions, except for the solution comprising the pro-polypeptide, i.e. the loading solution, applied to the immobilized metal ion affinity chromatography material in the different steps is independently of each other of from 1 to 20 column volumes, in one embodiment of from 1 to 10 column volumes.

In one embodiment the applying of the wash solution is for 3 to 20 column volumes. In one embodiment the applying of the wash solution is for 3 to 10 column volumes.

The methods as reported herein are exemplified in the following with a tetranectin-apolipoprotein A-I fusion protein as reported in WO2012/28526 and IGF-1 as reported in WO 2008/025527.

The tetranectin-apolipoprotein A-I fusion polypeptide comprises (in N- to C-terminal direction) the human tetranectin trimerising structural element and wild-type human apolipoprotein A-I. The amino acid sequence of the human tetranectin trimerising structural element can be shortened by the first 9 amino acids, thus, starting with the isoleucine residue of position 10, a naturally occurring truncation site. As a consequence of this truncation the O-glycosylation site at threonine residue of position 4 has been deleted. Between the tetranectin trimerising structural element and the human apolipoprotein A-I the five amino acid residues SLKGS were removed.

For improved expression and purification a construct can be generated comprising an N-terminal purification tag, e.g. a hexahistidine-tag, and a protease cleavage site for removal of the purification tag. In one embodiment the protease is IgA protease and the protease cleavage site is an IgA protease cleavage site. As a result of the specific cleavage of the protease some amino acid residues of the protease cleavage site are retained at the N-terminus of the polypeptide, i.e. in case of an IgA protease cleavage site two amino acid residues—as first alanine or glycine or serine or threonine and as second proline—are maintained at the N-terminus of the polypeptide, e.g. the tetranectin-apolipoprotein A-I fusion polypeptide.

The tetranectin trimerising structural element provides for a domain that allows for the formation of a tetranectin-apolipoprotein A-I homo-trimer that is constituted by non-covalent interactions between each of the individual tetranectin-apolipoprotein A-I monomers.

In one embodiment the apolipoprotein A-I fusion polypeptide is a variant comprising conservative amino acid substitutions.

In one embodiment the tetranectin-apolipoprotein A-I fusion polypeptide (interferon fragment and tag and cleavage site containing tetranectin-apolipoprotein A-I fusion polypeptide) comprises an expression and purification tag and has the amino acid sequence of

(SEQ ID NO: 01)
CDLPQTHSLGSHHHHHHGSVVAPPAPIVNAKKDVVNTKMFEELKSRLDTL
AQEVALLKEQQALQTVDEPPQSPWDRVKDLATVYVDVLKDSGRDYVSQF
EGSALGKQLNLKLLDNWDSVTSTFSKLREQLGPVTQEFWDNLEKETEGLR
QEMSKDLEEVKAKVQPYLDDFQKKWQEEMELYRQKVEPLRAELQEGAR
QKLHELQEKLSPLGEEMRDRARAHVDALRTHLAPYSDELRQRLAARLE
ALKENGGARLAEYHAKATEHLSTLSEKAKPALEDLRQGLLPVLESFKV
SFLSALEEYTKKLNTQ.

In one embodiment the tetranectin-apolipoprotein A-I fusion polypeptide (tag and cleavage site containing tetranectin-apolipoprotein A-I fusion polypeptide) comprises an expression and purification tag and has the amino acid sequence of

(SEQ ID NO: 02)
MRGSHHHHHHGSVVAPPAPIVNAKKDVVNTKMFEELKSRLDT
LAQEVALLKEQQALQTVDEPPQSPWDRVKDLATVYVDVLKDSG
RDYVSQFEGSALGKQLNLKLLDNWDSVTSTFSKLREQLGPVTQ
EFWDNLEKETEGLRQEMSKDLEEVKAKVQPYLDDFQKKWQEE
MELYRQKVEPLRAELQEGARQKLHELQEKLSPLGEEMRDRARA
HVDALRTHLAPYSDELRQRLAARLEALKENGGARLAEYHAKAT
EHLSTLSEKAKPALEDLRQGLLPVLESFKVSFLSALEEYTKKLNTQ.

In one embodiment the tetranectin-apolipoprotein A-I fusion polypeptide (tag and cleavage site containing tetranectin-apolipoprotein A-I fusion polypeptide) comprises an expression and purification tag and has the amino acid sequence of

(SEQ ID NO: 03)
HHHHHHGSVVAPPAPIVNAKKDVVNTKMFEELKSRLDTL
AQEVALLKEQQALQTVDEPPQSPWDRVKDLATVYVDVLK
DSGRDYVSQFEGSALGKQLNLKLLDNWDSVTSTFSKLR
EQLGPVTQEFWDNLEKETEGLRQEMSKDLEEVKAKVQPY
LDDFQKKWQEEMELYRQKVEPLRAELQEGARQKLHELQE
KLSPLGEEMRDRARAHVDALRTHLAPYSDELRQRLAARLE
ALKENGGARLAEYHAKATEHLSTLSEKAKPALEDLRQG
LLPVLESFKVSFLSALEEYTKKLNTQ.

It has to be noted that if a polypeptide is recombinantly produced in E. coli strains the N-terminal methionine residue is usually not efficiently cleaved off by E. coli proteases. Thus, the N-terminal methionine residue is partially present in the produced polypeptide.

The following examples, sequences and figures are provided to aid the understanding of the present invention, the true scope of which is set forth in the appended claims. It is understood that modifications can be made in the procedures set forth without departing from the spirit of the invention.

### FIGURES

FIG. 1 A) Elution diagram of an on-column cleaved tetranectin-apolipoprotein A-I pro-polypeptide without washing the IMAC column with a urea containing solution prior to the cleavage and elution under native conditions. The protein has been solubilized and applied to the column in a guanidinium chloride containing solution.
B) Magnification of the elution diagram of Figure A of the polypeptide. Subsequent washing with urea results in a sharp peak.

FIG. 2 Elution diagram of an on-column cleaved tetranectin-apolipoprotein A-I pro-polypeptide including the steps of washing with a guanidinium chloride containing solution, a urea containing solution and a buffered solution (Tris buffer) prior to the enzymatic on-column cleavage of the pro-polypeptide.

FIG. 3 Elution diagram of an on-column cleaved IGF-1 pro-polypeptide including the steps of washing with a guanidinium chloride containing solution, a urea containing solution and a buffered solution (Tris buffer) prior to the enzymatic on-column cleavage of the pro-polypeptide.

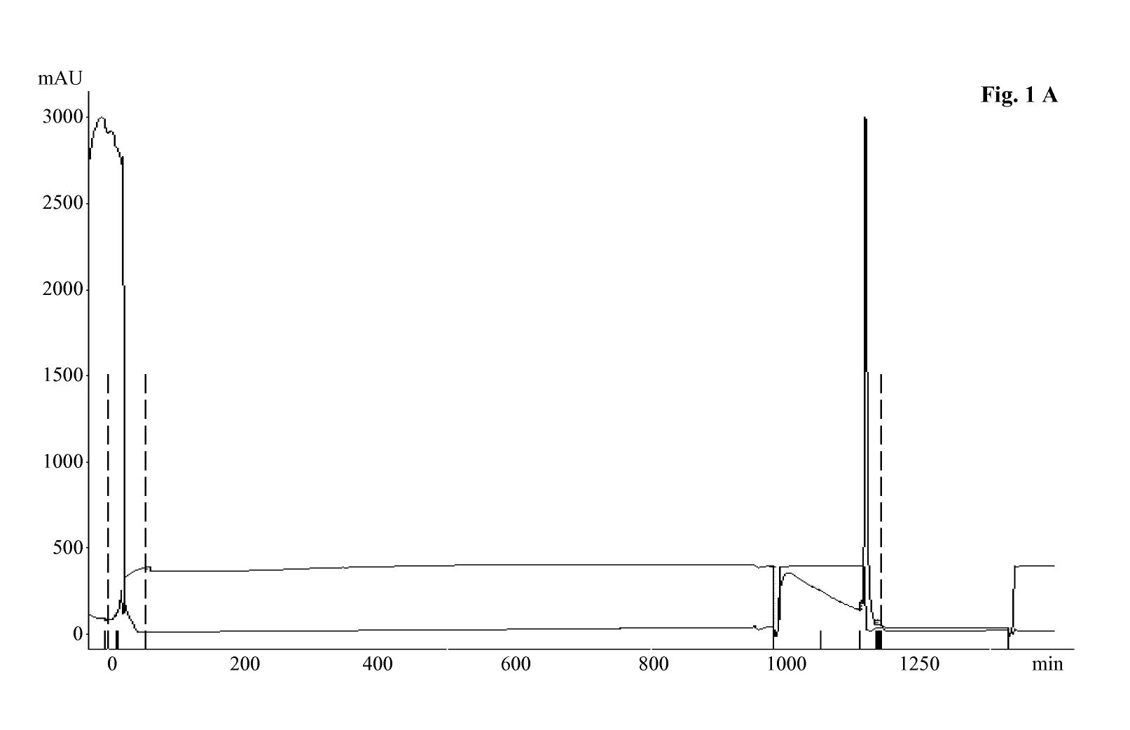

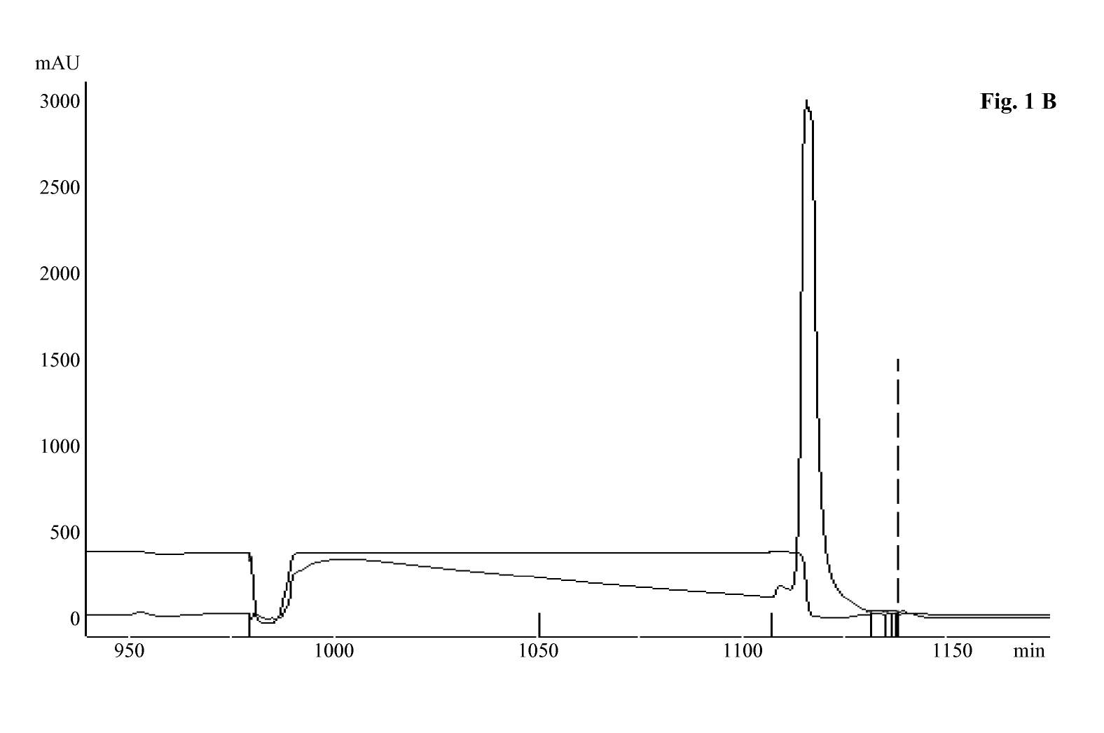

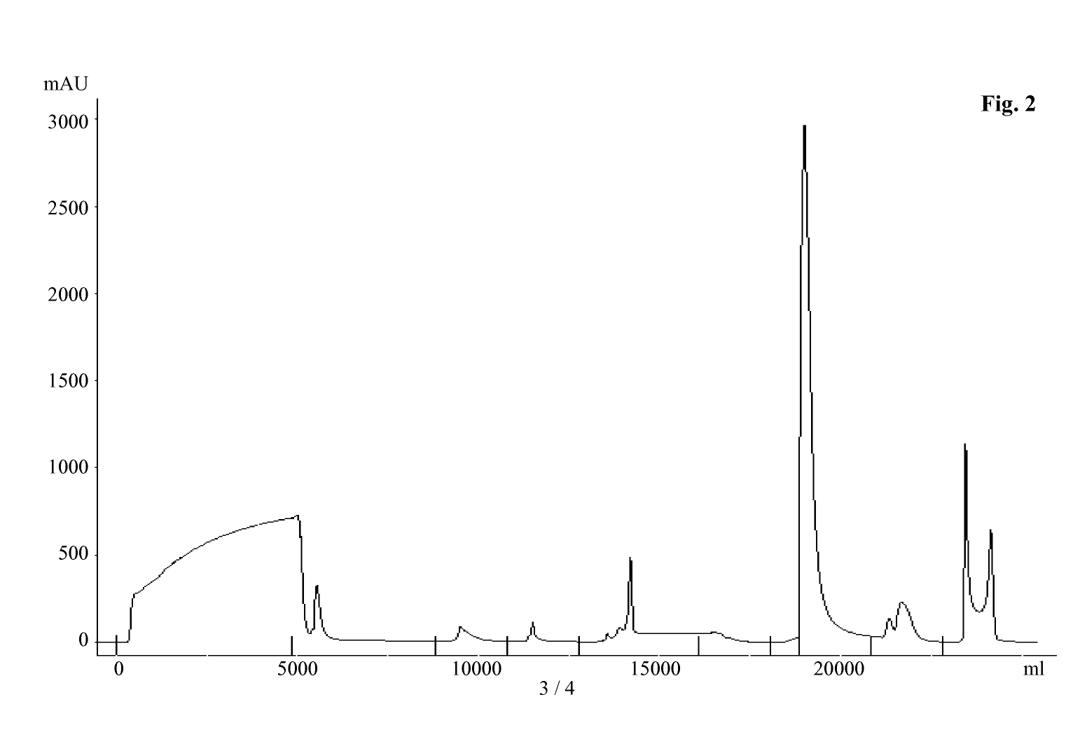

### EXAMPLES

### Materials and Methods

If not otherwise indicated the different chromatography methods have been performed according to the chromatography material manufacturer's manual.

### Recombinant DNA Techniques:

Standard methods were used to manipulate DNA as described in Sambrook, J., et al., Molecular Cloning: A Laboratory Manual; Cold Spring Harbor Laboratory Press, Cold Spring Harbor, N.Y. (1989). The molecular biological reagents were used according to the manufacturer's instructions.

### Protein Determination:

Protein concentration was determined by determining the optical density (OD) at 280 nm, with a reference wavelength of 320 nm, using the molar extinction coefficient calculated on the basis of the amino acid sequence.

### Size-Exclusion-HPLC:

The chromatography was conducted with a Tosoh Haas TSK 3000 SWXL column on an ASI-100 HPLC system (Dionex, Idstein, Germany). The elution peaks were monitored at 280 nm by a UV diode array detector (Dionex). After dissolution of the concentrated samples to 1 mg/ml the column was washed with a buffer consisting of 200 mM potassium dihydrogen phosphate and 250 mM potassium chloride pH 7.0 until a stable baseline was achieved. The analyzing runs were performed under isocratic conditions using a flow rate of 0.5 ml/min. over 30 min. at room temperature. The chromatograms were integrated manually with Chromeleon (Dionex, Idstein, Germany).

### Reversed Phase HPLC (RP-HPLC):

The purity is analyzed by RP-HPLC. The assay is performed on a Phenomenex C18 column using an acetonitrile/aqueous TFA gradient. The elution profile is monitored as UV absorbance at 215 nm. The percentages of the eluted substances are calculated based upon the total peak area of the eluted proteins.

### DNA-Threshold-System:

See e.g. Merrick, H., and Hawlitschek, G., Biotech Forum Europe 9 (1992) 398-403.

### Host Cell Protein Determination:

The walls of the wells of a micro titer plate are coated with a mixture of serum albumin and Streptavidin. A goat derived polyclonal antibody against HCP is bound to the walls of the wells of the micro titer plate. After a washing step different wells of the micro titer plate are incubated with a HCP calibration sequence of different concentrations and sample solution. After the incubation not bound sample material is removed by washing with buffer solution. For the detection the wells are incubated with an antibody peroxidase conjugate to detect bound host cell protein. The fixed peroxidase activity is detected by incubation with ABTS and detection at 405 nm.

### DNA Determination:

Biotin was bound to a microtiter plate. A reaction mixture of streptavidin, single-stranded DNA and biotinylated single-stranded DNA binding protein was added. The binding protein was able to bind DNA and was biotinylated. In this manner it was possible to specifically remove the DNA from the sample mixture. The streptavidin bound the biotin on the microtiter plate as well as the biotin which was coupled to the single-stranded DNA binding protein. A DNA-specific antibody which was coupled to urease was added to this total complex. Addition of urea resulted in a hydrolysis of the urea which caused a local change in the pH. This change can be detected as an altered surface potential. The change in the surface potential was proportional to the amount of bound DNA. Single stranded DNA was obtained by proteinase K digestion and denaturation with SDS.

General Method for the Isolation, Solubilization and Re-Folding of Polypeptide from Inclusion Bodies:

In addition to the method performed in the cited literature can the preparation of inclusion bodies e.g. be performed according the method by Rudolph, et al. (Rudolph, R., et al., Folding Proteins, In: Creighton, T.E., (ed.): Protein function: A Practical Approach, Oxford University Press (1997) 57-99). The inclusion bodies were stored at −70° C. Solubilization of the inclusion bodies can likewise be performed according the method by Rudolph, et al. (Rudolph, R., et al., Folding Proteins, In: Creighton, T.E., (ed.): Protein function: A Practical Approach, Oxford University Press (1997) 57-99).

### Example 1

### Making and Description of the E. coli Expression Plasmids

The tetranectin-apolipoprotein A-I pro-polypeptide was prepared by recombinant means. The amino acid sequence of the expressed pro-polypeptide in N- to C-terminal direction is as follows:
the amino acid methionine (M),
a fragment of an interferon sequence that has the amino acid sequence of CDLPQTHSL (SEQ ID NO: 19),
a GS linker,
a hexa-histidine tag that has the amino acid sequence of HHHHHH (SEQ ID NO: 20),
a GS linker,
an IgA protease cleavage site that has the amino acid sequence of VVAPPAP (SEQ ID NO: 11), and
a tetranectin-apolipoprotein A-I that has the amino acid sequence of SEQ ID NO: 04.

The tetranectin-apolipoprotein A-I pro-polypeptides as described above are precursor polypeptides from which the mature tetranectin-apolipoprotein A-I fusion polypeptide was released by enzymatic on-column cleavage in vitro using IgA protease.

The pro-polypeptide encoding fusion gene was assembled with known recombinant methods and techniques by connection of appropriate nucleic acid segments. Nucleic acid sequences made by chemical synthesis were verified by DNA sequencing. The expression plasmid for the production of tetranectin-apolipoprotein A-I pro-polypeptide of SEQ ID NO: 01 encoding a fusion polypeptide of SEQ ID NO: 04 was prepared as follows.

Making of the E. coli Expression Plasmid

Plasmid 4980 (4980-pBRori-URA3-LACI-SAC) is an expression plasmid for the expression of core-streptavidin in E. coli. It was generated by ligation of the 3142 bp long EcoRI/CelII-vector fragment derived from plasmid 1966 (1966-pBRori-URA3-LACI-T-repeat; reported in EP-B 1 422 237) with a 435 bp long core-streptavidin encoding EcoRI/CelII-fragment.

The core-streptavidin E. coli expression plasmid comprises the following elements:
the origin of replication from the vector pBR322 for replication in E. coli (corresponding to by position 2517-3160 according to Sutcliffe, G., et al., Quant. Biol. 43 (1979) 77-90),
the URA3 gene of Saccharomyces cerevisiae coding for orotidine 5′-phosphate decarboxylase (Rose, M. et al. Gene 29 (1984) 113-124) which allows plasmid selection by complementation of E. coli pyrF mutant strains (uracil auxotrophy),
the core-streptavidin expression cassette comprising
the T5 hybrid promoter (T5-PN25/03/04 hybrid promoter according to Bujard, H., et al. Methods. Enzymol. 155 (1987) 416-433 and Stueber, D., et al., Immunol. Methods IV (1990) 121-152) including a synthetic ribosomal binding site according to Stueber, D., et al. (see before),
the core-streptavidin gene,
two bacteriophage-derived transcription terminators, the λ-T0 terminator (Schwarz, E., et al., Nature 272 (1978) 410-414) and the fd-terminator (Beck E. and Zink, B. Gene 1-3 (1981) 35-58),
the lacI repressor gene from E. coli (Farabaugh, P. J., Nature 274 (1978) 765-769).

The final expression plasmid for the expression of the tetranectin-apolipoprotein A-I pro-polypeptide was prepared by excising the core-streptavidin structural gene from vector 4980 using the singular flanking EcoRI and CelII restriction endonuclease cleavage site and inserting the EcoRII/CelII restriction site flanked nucleic acid encoding the precursor polypeptide into the 3142 bp long EcoRI/CelII-4980 vector fragment.

### Example 2

### Expression of Tetranectin-Apolipoprotein A-I Pro-Polypeptide

For the expression of the pro-polypeptide there was employed an E. coli host/vector system which enables an antibiotic-free plasmid selection by complementation of an E. coli auxotrophy (PyrF) (see EP 0972838 and U.S. Pat. No. 6,291,245).

The E. coli K12 strain CSPZ-2 (leuB, proC, trpE, th-1, ΔpyrF) was transformed by electroporation with the expression plasmid p(IFN-His6-IgA-tetranectin-apolipoprotein A-I). The transformed E. coli cells were first grown at 37° C. on agar plates.

### Fermentation Protocol 1:

For pre-fermentation a M9 medium according to Sambrook et al (Molecular Cloning: A laboratory manual. Cold Spring Harbor Laboratory Press; 2nd edition (December 1989) supplemented with about 1 g/l L-leucine, about 1 g/l L-proline and about 1 mg/l thiamine-HCl has been used.

For pre-fermentation 300 ml of M9-medium in a 1000 ml Erlenmeyer-flask with baffles was inoculated with 2 ml out of a primary seed bank ampoule. The cultivation was performed on a rotary shaker for 13 hours at 37° C. until an optical density (578 nm) of 1-3 was obtained.

For fermentation a batch medium according to Riesenberg et al. was used (Riesenberg, D., et al., J. Biotechnol. 20 (1991) 17-27): 27.6 g/l glucose*H₂O, 13.3 g/l KH₂PO₄, 4.0 g/l (NH₄)₂HPO₄, 1.7 g/l citrate, 1.2 g/l MgSO₄*7 H₂O, 60 mg/l iron(III)citrate, 2.5 mg/l CoCl₂*6 H₂O, 15 mg/l MnCl₂*4 H₂O, 1.5 mg/l CuCl₂*2 H₂O, 3 mg/l H₃BO₃, 2.5 mg/l Na₂MoO₄*2 H₂O, 8 mg/l Zn(CH₃C00)₂*2 H₂O, 8.4 mg/l Titriplex III, 1.3 ml/l Synperonic 10% anti foam agent. The batch medium was supplemented with 5.4 mg/l Thiamin-HCl and 1.2 g/l L-leucine and L-proline respectively. The feed 1 solution contained 700 g/l glucose supplemented with 19.7 g/l MgSO₄*7 H₂O. The alkaline solution for pH regulation was an aqueous 12.5% (w/v) NH₃ solution supplemented with 50 g/l L-leucine and 50 g/l L-proline respectively. All components were dissolved in deionized water.

The fermentation was carried out in a 10 l Biostat C DCU3 fermenter (Sartorius, Melsungen, Germany). Starting with 6.4 l sterile fermentation batch medium plus 300 ml inoculum from the pre-fermentation the batch fermentation was performed at 37° C., pH 6.9±0.2, 500 mbar and an aeration rate of 10 l/min. After the initially supplemented glucose was depleted the temperature was shifted to 28° C. and the fermentation entered the fed-batch mode. Here the relative value of dissolved oxygen (pO2) was kept at 50% (DO-stat, see e.g. Shay, L. K., et al., J. Indus. Microbiol. Biotechnol. 2 (1987) 79-85) by adding feed 1 in combination with constantly increasing stirrer speed (550 rpm to 1000 rpm within 10 hours and from 1000 rpm to 1400 rpm within 16 hours) and aeration rate (from 10 l/min to 16 l/min in 10 hours and from 16 l/min to 20 l/min in 5 hours). The supply with additional amino acids resulted from the addition of the alkaline solution, when the pH reached the lower regulation limit (6.70) after approximately 8 hours of cultivation. The expression of recombinant therapeutic protein was induced by the addition of 1 mM IPTG at an optical density of 70.

Samples drawn from the fermenter, one prior to induction and the others at dedicated time points after induction of protein expression are analyzed with SDS-Polyacrylamide gel electrophoresis. From every sample the same amount of cells (OD_{Target}=5) are resuspended in 5 mL PBS buffer and disrupted via sonication on ice. Then 100 μL of each suspension are centrifuged (15,000 rpm, 5 minutes) and each supernatant is withdrawn and transferred to a separate vial. This is to discriminate between soluble and insoluble expressed target protein. To each supernatant (=soluble) fraction 300 μL and to each pellet (=insoluble) fraction 400 μL of SDS sample buffer (Laemmli, U.K., Nature 227 (1970) 680-685) are added. Samples are heated for 15 minutes at 95° C. under shaking to solubilize and reduce all proteins in the samples. After cooling to room temperature 5 μL of each sample are transferred to a 4-20% TGX Criterion Stain Free polyacrylamide gel (Bio-Rad). Additionally 5 μl molecular weight standard (Precision Plus Protein Standard, Bio-Rad) and 3 amounts (0.3 μl, 0.6 μl and 0.9 μl) quantification standard with known product protein concentration (0.1 μg/μl) are positioned on the gel.

The electrophoresis was run for 60 Minutes at 200 V and thereafter the gel was transferred the GelDOC EZ Imager (Bio-Rad) and processed for 5 minutes with UV radiation. Gel images were analyzed using Image Lab analysis software (Bio-Rad). With the three standards a linear regression curve was calculated with a coefficient of >0.99 and thereof the concentrations of target protein in the original sample was calculated.

At the end of fermentation the cytoplasmatic and soluble expressed tetranectin-apolipoprotein A-I pro-polypeptide was transferred to insoluble protein aggregates, the so called inclusion bodies, with a heat step where the whole culture broth in the fermenter is heated to 50° C. for 1 or 2 hours before harvest (see e.g. EP-B 1 486 571). Thereafter, the content of the fermenter was centrifuged with a flow-through centrifuge (13,000 rpm, 13 l/h) and the harvested biomass was stored at −20° C. until further processing. The synthesized tetranectin-apolipoprotein A-I pro-polypeptide were found exclusively in the insoluble cell debris fraction in the form of insoluble protein aggregates, so-called inclusion bodies (IBs).

### Fermentation Protocol 2:

For pre-fermentation a M9 medium according to Sambrook et al. (Molecular Cloning: A laboratory manual. Cold Spring Harbor Laboratory Press; 2nd edition (December 1989)) supplemented with about 1 g/l L-leucine, about 1 g/l L-proline and about 1 mg/l thiamine-HCl has been used.

For pre-fermentation 300 ml of modified M9-medium in a 1000 ml Erlenmeyer-flask with baffles was inoculated from agar plate or with 1-2 ml out of a primary seed bank ampoule. The cultivation was performed on a rotary shaker for 13 hours at 37° C. until an optical density (578 nm) of 1-3 was obtained.

For fermentation and high yield expression of tetranectin-apolipoprotein A-I the following batch medium and feeds were used:

8.85 g/l glucose, 63.5 g/l yeast extract, 2.2 g/l NH₄Cl, 1.94 g/l L-leucine, 2.91 g/l L-proline, 0.74 g/l L-methionine, 17.3 g/l KH₂PO₄*H2_{O}, 2.02 g/l MgSO₄*7 H₂O, 25.8 mg/l Thiamin-HCl, 1.0 ml/l Synperonic 10% anti foam agent. The feed 1 solution contained 333 g/l yeast extract and 333 g/l 85%-glycerol supplemented with 1.67 g/l L-methionine and 5 g/l L-leucine and L-proline each. The feed 2 was a solution of 600 g/l L-Proline. The alkaline solution for pH regulation was a 10% (w/v) KOH solution and as acid a 75% glucose solution was used. All components were dissolved in deionized water.

The fermentation was carried out in a 10 l Biostat C DCU3 fermenter (Sartorius, Melsungen, Germany). Starting with 5.15 l sterile fermentation batch medium plus 300 ml inoculum from the pre-fermentation the fed-batch fermentation was performed at 25° C., pH 6.7±0.2, 300 mbar and an aeration rate of 10 l/min. Before the initially supplemented glucose was depleted the culture reached an optical density of 15 (578 nm) and the fermentation entered the fed-batch mode when feed 1 was started with 70 g/h. Monitoring the glucose concentration in the culture the feed 1 was increased to a maximum of 150 g/h while avoiding glucose accumulation and keeping the pH near the upper regulation limit of 6.9. At an optical density of 50 (578 nm) feed 2 was started with a constant feed rate of 10 ml/h. The relative value of dissolved oxygen (pO₂) was kept above 50% by increasing stirrer speed (500 rpm to 1500 rpm), aeration rate (from 10 l/min to 20 l/min) and pressure (from 300 mbar to 500 mbar) in parallel. The expression of recombinant therapeutic protein was induced by the addition of 1 mM IPTG at an optical density of 90.

Seven samples drawn from the fermenter, one prior to induction and the others at dedicated time points after induction of protein expression are analyzed with SDS-Polyacrylamide gel electrophoresis. From every sample the same amount of cells (OD_{Target}=5) are resuspended in 5 mL PBS buffer and disrupted via sonication on ice. Then 100 μL of each suspension are centrifuged (15,000 rpm, 5 minutes) and each supernatant is withdrawn and transferred to a separate vial. This is to discriminate between soluble and insoluble expressed target protein. To each supernatant (=soluble) fraction 300 μL and to each pellet (=insoluble) fraction 200 μL of SDS sample buffer (Laemmli, U.K., Nature 227 (1970) 680-685) are added. Samples are heated for 15 minutes at 95° C. under shaking to solubilize and reduce all proteins in the samples. After cooling to room temperature 5 μL of each sample are transferred to a 10% Bis-Tris polyacrylamide gel (Novagen). Additionally 5 μl molecular weight standard (Precision Plus Protein Standard, Bio-Rad) and 3 amounts (0.3 μl, 0.6 μl and 0.9 μl) quantification standard with known product protein concentration (0.1 μg/μl) are positioned on the gel.

The electrophoresis was run for 35 minutes at 200 V and then the gel was stained with Coomassie Brilliant Blue R dye, destained with heated water and transferred to an optical densitometer for digitalization (GS710, Bio-Rad). Gel images were analyzed using Quantity One 1-D analysis software (Bio-Rad). With the three standards a linear regression curve is calculated with a coefficient of >0.98 and thereof the concentrations of target protein in the original sample was calculated.

At the end of fermentation the cytoplasmatic and soluble expressed tetranectin-apolipoprotein A-I pro-polypeptide was transferred to insoluble protein aggregates, the so called inclusion bodies (IBs), with a heat step where the whole culture broth in the fermenter is heated to 50° C. for 1 or 2 hours before harvest (see e.g. EP-B 1 486 571). After the heat step the synthesized tetranectin-apolipoprotein A-I pro-polypeptide were found exclusively in the insoluble cell debris fraction in the form of IBs.

The contents of the fermenter are cooled to 4-8° C., centrifuged with a flow-through centrifuge (13,000 rpm, 13 l/h) and the harvested biomass is stored at −20° C. until further processing. The total harvested biomass yield ranged between 39 g/l and 90 g/l dry matter depending on the expressed construct.

### Example 3

### Inclusion Body Preparation of Tetranectin-Apolipoprotein A-I Pro-Polypeptide

Inclusion body preparation was carried out by resuspension of harvested bacteria cells in a potassium phosphate buffered solution or a Tris buffered solution (0.1 M, supplemented with 1 mM MgSO₄, pH 6.5). After the addition of DNAse the cell were disrupted by homogenization at a pressure of 900 bar. A buffer solution comprising 1.5 M NaCl and 60 mM EDTA was added to the homogenized cell suspension. After the adjustment of the pH value to 5.0 with 25% (w/v) HCl the final inclusion body slurry was obtained after a further centrifugation step. The slurry was stored at −20° C. in single use, sterile plastic bags until further processing.

### Example 4a

### IB Solubilization of His-Tagged Tetranectin-Apolipoprotein A-I Pro-Polypeptide Under Denaturing Conditions

The inclusion body slurry from Example 3 was solubilized with guanidinium chloride (GdmCl) at a final GdmCl concentration of 6 M. After solubilization, the slurry was filtered by a combination of depth filters. This solution of denatured tetranectin-apolipoprotein A-I pro-polypeptide was diluted 3-fold to obtain a final GdmCl concentration of 2 M in 50 mM sodium phosphate buffer, pH 8.0 and again filtered to get a clear solution suitable for the following chromatography step (see Example 5).

### Example 4b

### IB Solubilization of His-Tagged Tetranectin-Apolipoprotein A-I Pro-Polypeptide Under Non-Denaturing Conditions

The inclusion body slurry from Example 3 was solubilized with potassium hydroxide solution about 30 mM (adjusted to pH 11.5) for one hour. Thereafter the pH value is adjusted to pH 8 and the slurry was filtered by a combination of depth filters. The filtrate is suitable for the following chromatography step (see Example 5).

### Example 5a

### Loading of Denatured His-Tagged Tetranectin-Apolipoprotein A-I Pro-Polypeptide onto an IMAC Column

The solution of Example 4a was loaded onto an immobilized metal ion affinity chromatography (IMAC; Fractogel® EMD Chelate, cat. no. 110338, Merck, Darmstadt, Germany, 230 ml column volume, 24 cm bead height, 3.5 cm diameter) column preloaded with Zn²⁺ ions and equilibrated with the denaturing loading buffer (2 M GdmCl, 50 mM sodium phosphate, pH 8.0, four column volumes) at a tetranectin-apolipoprotein A-I pro-polypeptide load of 15 to 20 mg per ml of packed gel bed. The binding of the protein to the column was effected by the interaction of the His-tag with the immobilized Zn²⁺ ions.

### Example 5b

### Loading of Native His-Tagged Tetranectin-Apolipoprotein A-I Pro-Polypeptide onto an IMAC Column

The solution of Example 4b was loaded onto an immobilized metal ion affinity chromatography (IMAC; Fractogel® EMD Chelate, cat. no. 110338, Merck, Darmstadt, Germany, 230 ml column volume, 24 cm bead height, 3.5 cm diameter) column preloaded with Zn²⁺ ions and equilibrated with the native loading buffer (30 mM sodium phosphate, pH 8.0, four column volumes) at a tetranectin-apolipoprotein A-I pro-polypeptide load of 15 to 20 mg per ml of packed gel bed. The binding of the protein to the column was effected by the interaction of the His-tag with the immobilized Zn²⁺ ions.

### Example 6

### Washing of the His-Tagged Tetranectin-Apolipoprotein A-I Pro-Polypeptide Loaded IMAC Column

After loading, the immobilized metal ion affinity chromatography material containing column was washed with 4 to 6 column volumes of the denaturing loading buffer (2 M GdmCl, 50 mM sodium phosphate, pH 8.0) in order to remove non-specifically bound proteins and other contaminants under denaturing conditions.

This wash step was followed by a second wash step under denaturing conditions with 4 M urea dissolved in 250 mM Tris buffer, pH 8.0, for three column volumes in order to remove remaining GdmCl from the column.

The urea wash step was immediately (to avoid carbamoylation of the bound protein) followed by a wash step under native conditions to remove the denaturing urea and allow renaturation of the IMAC-bound tetranectin-apolipoprotein A-I pro-polypeptide (renaturation of the bound protein is a prerequisite for the following proteolytic cleavage & native elution). The native wash step was performed with e.g. 1 M Tris buffer, pH 8.0, for at least three column volumes. This wash step may be followed by other native wash steps containing in addition to the 1 M Tris e.g. imidazole and/or arginine (an exemplary Tris/imidazole/arginine buffer has the composition of 1 M Tris, 70 mM imidazole and 180 mM arginine) to further remove non-specifically bound contaminants followed by a final wash step with 1 to 1.2 M Tris, pH 8.0. The concentration of the e.g. imidazole and/or arginine should be chosen in a way not to elute the bound protein from the immobilized metal ion affinity chromatography material.

### Example 7

### On-Column-Cleavage of the His-Tagged Tetranectin-Apolipoprotein A-I Pro-Polypeptide and Elution of Tetranectin-Apolipoprotein A-I

IgA protease (EC 3.4.24.13) was dissolved in the final native washing buffer (1 to 1.2 M Tris, pH 8.0) at a concentration of 1:500 in comparison to the originally loaded His-tagged tetranectin-apolipoprotein A-I pro-polypeptide (i.e. for example 30 μg/ml IgA protease in case of a tetranectin-apolipoprotein A-I load of 15 mg/ml). From this solution, 1 column volume was loaded onto the column followed by a flow stop of at least 12 hours to allow the protease to cleave the column-bound pro-protein, i.e. to cleave off the His-tag and thereby release the tetranectin-apolipoprotein A-I from the column.

After the protease incubation, the tetranectin-apolipoprotein A-I cleaved from the His-tag was eluted by washing the column with the native washing buffer (1 to 1.2 M Tris, pH 8.0).

The yield of cleaved and recovered polypeptide is about 60% to 75% with respect to the applied pro-polypeptide.

The eluted tetranectin-apolipoprotein A-I obtained by the examples described can be further purified by additional chromatographic steps and then be lipidated as described e.g. in WO2012/28524.

In the following table the results obtained for one exemplary run are summarized.

TABLE

| | Amount | Amount | |
|---|---|---|---|
| | bound | recovered | |
| | on | form | |
| | column | column | yield |
| step | [mg] | [mg] | [%] |
| load | 7748 | n.a. | 100 |
| IMAC | | | |
| eluate | n.a. | 5546 | 72 |
| IMAC | | | |

n.a. = not applicable

### Example 8

### Washing of the His-Tagged Tetranectin-Apolipoprotein A-I Pro-Polypeptide Loaded IMAC Column Omitting the Urea Wash Step as Described in Example 6 and on-Column-Cleavage of the His-Tagged Tetranectin-Apolipoprotein A-I Pro-Polypeptide and Elution of Tetranectin-Apolipoprotein A-I

After loading under denaturing conditions according to Example 5a, the immobilized metal ion affinity chromatography material containing column was washed with 4 to 6 column volumes of the denaturing loading buffer (2 M GdmCl, 50 mM sodium phosphate, pH 8.0) in order to remove non-specifically bound proteins and other contaminants under denaturing conditions.

The GdmCl wash step was followed by a wash step under native conditions to remove the denaturing GdmCl and allow renaturation of the IMAC-bound tetranectin-apolipoprotein A-I pro-polypeptide (renaturation of the bound protein is a prerequisite for the following proteolytic cleavage & native elution). The native wash step was performed with e.g. 1 M Tris buffer, pH 8.0, for at least three column volumes. This wash step may be followed by other native wash steps containing in addition to the 1 M Tris e.g. imidazole and/or arginine (an exemplary Tris/imidazole/arginine buffer has the composition of 1 M Tris, 70 mM imidazole and 180 mM arginine) to further remove non-specifically bound contaminants followed by a final wash step with 1 to 1.2 M Tris, pH 8.0. The concentration of the e.g. imidazole and/or arginine should be chosen in a way not to elute the bound protein from the immobilized metal ion affinity chromatography material.

IgA protease (EC 3.4.24.13) was dissolved in the final native washing buffer (1 to 1.2 M Tris, pH 8.0) at a concentration of 1:500 in comparison to the originally loaded His-tagged tetranectin-apolipoprotein A-I pro-polypeptide (i.e. for example 30 μg/ml IgA protease in case of a tetranectin-apolipoprotein A-I load of 15 mg/ml). From this solution, 1 column volume was loaded onto the column followed by a flow stop of at least 12 hours to allow the protease to cleave the column-bound pro-protein, i.e. to cleave off the His-tag and thereby release the tetranectin-apolipoprotein A-I from the column.

After the protease incubation, the tetranectin-apolipoprotein A-I cleaved from the His-tag was eluted by washing the column with the native washing buffer (1 to 1.2 M Tris, pH 8.0).

The yield of cleaved and recovered polypeptide when omitting the urea wash step is less than 50% even after eluting with about 20 column volumes of the native washing buffer with respect to the applied pro-polypeptide. The protein concentration in the eluate under these conditions is very low (about 0.2 mg/ml) in comparison to the eluate obtained under the conditions as described in example 7 (about 2 mg/ml).

When performing the urea wash step after eluting tetranectin-apolipoprotein A-I with the native wash buffer another about 40% cleaved polypeptide can be recovered at a concentration of about 0.9 mg/ml.

### Example 9

### Inclusion Body Preparation of IGF-1 Pro-Polypeptide

Inclusion body preparation was carried out by resuspension of harvested bacteria cells in a potassium phosphate buffered solution or a Tris buffered solution (0.1 M, supplemented with 1 mM MgSO₄, pH 6.5). After the addition of DNAse the cell were disrupted by homogenization at a pressure of 900 bar. A buffer solution comprising 1.5 M NaCl and 60 mM EDTA was added to the homogenized cell suspension. After the adjustment of the pH value to 5.0 with 25% (w/v) HCl the final inclusion body slurry was obtained after a further centrifugation step. The slurry was stored at −20° C. in single use, sterile plastic bags until further processing.

### Example 10

### IB Solubilization of His-Tagged IGF-1 Pro-Polypeptide Under Denaturing Conditions

40 g of the inclusion body slurry from Example 9 was solubilized with a solubilization buffer (6.7 M guanidinium chloride (GdmCl), 1 mM EDTA, 10 mM sodium acetate, pH 4.0) at a final GdmCl concentration of 6 M. After solubilization, the slurry was filtered by a combination of depth filters. This solution of denatured IGF-1 pro-polypeptide was diluted 3-fold to obtain a final GdmCl concentration of 2 M in 50 mM sodium phosphate buffer, pH 8.0 and again filtered to get a clear solution of about 400 ml final volume.

### Example 11

### Renaturing of Denatured His-Tagged IGF-1 Pro-Polypeptide

400 ml solubilized IGF-1 pro polypeptide were added to 3.6 l of renaturing buffer (1 M arginine, 2 mM GSH, 0.1 mM GSSG, pH 8.0) during a time period of 3 hours. The IGF-1 pro-polypeptide is renatured for 5 hours with stirring and thereafter 12 hours without stirring. After renaturing was complete the solution was diafiltered against 7times its volume of equilibration buffer of the following IMAC chromatography (2 M guanidinium hydrochloride, 50 mM sodium phosphate, pH 8.0) resulting in a total volume of 21.

### Example 12

### Loading of Denatured His-Tagged IGF-1 Pro-Polypeptide onto an IMAC Column

11 of the solution of Example 11 was loaded onto an immobilized metal ion affinity chromatography (IMAC; Fractogel® EMD Chelate, cat. no. 110338, Merck, Darmstadt, Germany, 230 ml column volume, 24 cm bead height, 3.5 cm diameter) column preloaded with Zn² ions and equilibrated with the denaturing loading buffer (2 M GdmCl, 50 mM sodium phosphate, pH 8.0, two column volumes). The binding of the protein to the column was effected by the interaction of the His-tag with the immobilized Zn²⁺ ions.

### Example 13

### Washing of the His-Tagged IGF-1 Pro-Polypeptide Loaded IMAC Column

After loading, the immobilized metal ion affinity chromatography material containing column was washed with 10 column volumes of the denaturing loading buffer (2 M GdmCl, 50 mM sodium phosphate, pH 8.0) in order to remove non-specifically bound proteins and other contaminants under denaturing conditions.

This wash step was followed by a second wash step under denaturing conditions with 4 M urea dissolved in 250 mM Tris buffer, pH 8.0, for three column volumes in order to remove remaining GdmCl from the column.

The urea wash step was immediately (to avoid carbamoylation of the bound protein) followed by a wash step under native conditions to remove the denaturing urea and allow renaturation of the IMAC-bound tetranectin-apolipoprotein A-I pro-polypeptide (renaturation of the bound protein is a prerequisite for the following proteolytic cleavage & native elution). The native wash step was performed with e.g. 1 M Tris buffer, pH 8.0, for four column volumes. This wash step was followed by other native wash steps containing in addition to the 1 M Tris e.g. imidazole and/or arginine (an exemplary Tris/imidazole/arginine buffer has the composition of 1 M Tris, 70 mM imidazole and 180 mM arginine, pH 8.0) to further remove non-specifically bound contaminants followed by a final wash step with 1.2 M Tris, pH 8.0.

### Example 14

### On-Column-Cleavage of the His-Tagged IGF-1 Pro-Polypeptide and Elution of IGF-1

IgA protease (EC 3.4.24.13) was dissolved in the final native washing buffer (1.2 M Tris, pH 8.0) at a concentration of 1:500 in comparison to the originally loaded His-IGF-1 pro-polypeptide. From this solution, 1.1 column volumes was loaded onto the column followed by a flow stop of 12 hours to allow the protease to cleave the column-bound pro-protein, i.e. to cleave off the His-tag and thereby release the IGF-1 from the column.

After the protease incubation, the IGF-1 cleaved from the His-tag was eluted by washing the column with the native washing buffer (1.2 M Tris, pH 8.0).

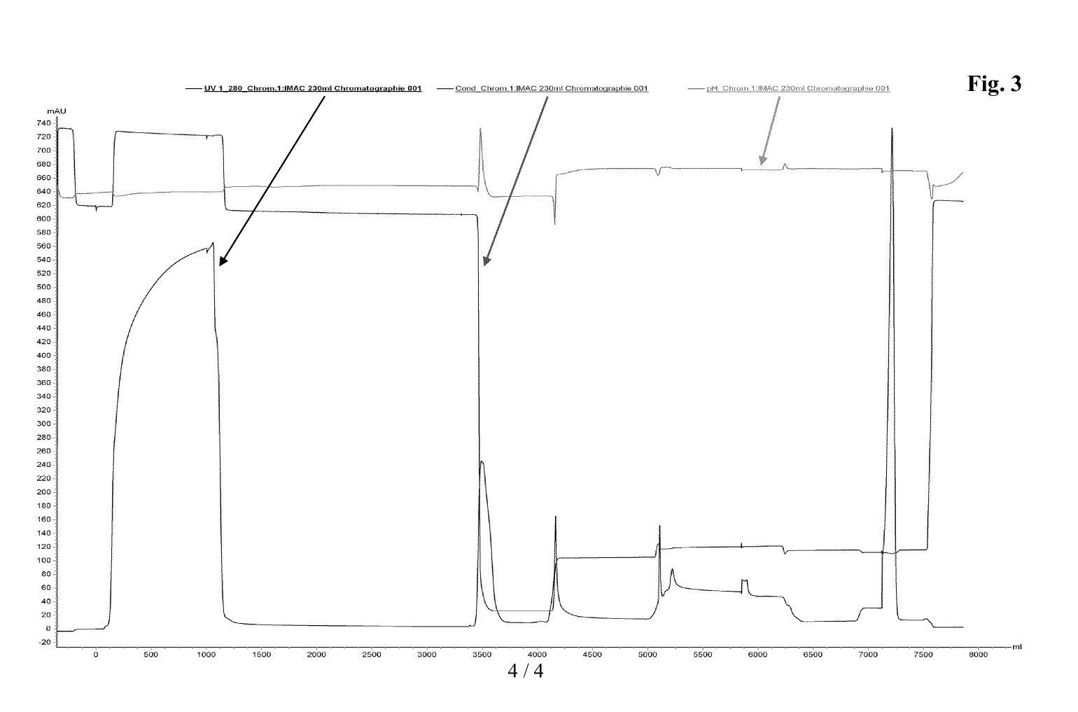

The respective chromatogram is shown in FIG. 3.

1. A method for producing a polypeptide from a pro-polypeptide, whereby the pro-polypeptide comprises at its N- or C-terminus a metal ion affinity chromatography tag and a protease cleavage site located between the tag and the polypeptide, by an on-column enzymatic cleavage of the protease cleavage site on an immobilized metal ion affinity chromatography column comprising the following steps:
denaturing the pro-polypeptide bound to the metal ion affinity chromatography material,
renaturing the pro-polypeptide bound to the metal ion affinity chromatography material, and
incubating the bound pro-polypeptide with a protease and thereby producing the polypeptide.

2. A method for producing a polypeptide from a pro-polypeptide, whereby the pro-polypeptide comprises at its N- or C-terminus a metal ion affinity chromatography tag and a protease cleavage site located between the tag and the polypeptide, by an on-column enzymatic cleavage of the protease cleavage site on an immobilized metal ion affinity chromatography column comprising the following steps:
contacting the bound pro-polypeptide with a solution comprising a denaturing agent,
optionally contacting the bound pro-polypeptide with a solution comprising urea or a urea derivatives if the solution comprising a denaturing agent employed in the previous step was free of urea or a urea derivative or contacting the bound pro-polypeptide with a solution comprising urea or a urea derivatives if the solution comprising a denaturing agent employed in the previous step comprised a mixture of urea or a urea derivative and a denaturing agent, and
recovering the polypeptide from the immobilized metal ion affinity chromatography column by incubating the bound pro-polypeptide with a protease and thereby producing a polypeptide.

3. The method according to claim 1, additionally comprising: a washing step directly prior to incubation of the protease with a solution free of denaturing agents.

4-15. (canceled)

16. The method according to claim 2, additionally comprising: a washing step directly prior to incubation of the protease with a solution free of denaturing agents.

17. The method according to claim 1, wherein the polypeptide is recovered in native form.

18. The method according to claim 2, wherein the polypeptide is recovered in native form.

19. The method according to claim 3, wherein the polypeptide is recovered in native form.

20. The method according to claim 16, wherein the polypeptide is recovered in native form.

21. The method according to claim 1, wherein the polypeptide is recovered in denatured form.

22. The method according to claim 2, wherein the polypeptide is recovered in denatured form.

23. The method according to claim 3, wherein the polypeptide is recovered in denatured form.

24. The method according to claim 16, wherein the polypeptide is recovered in denatured form.

25. The method according to claim 1, wherein the denaturing agent is selected from the group guanidinium chloride, urea, urea derivative, thiourea, tetramethylurea, or combinations thereof.

26. The method according to claim 25, wherein the urea or urea derivative has a concentration of from 0.5M to 8M.

27. The method according to claim 2, wherein the denaturing agent is selected from the group guanidinium chloride, urea, urea derivative, thiourea, tetramethylurea, or combinations thereof.

28. The method according to claim 25, wherein the denaturing agent has a concentration of from 0.5 M to 6 M.

29. The method according to claim 27, wherein the denaturing agent has a concentration of from 0.5 M to 6 M.

30. The method according to claim 1, wherein the pro-polypeptide is applied in native form or in denatured form to the immobilized metal ion affinity chromatography material.

31. The method according to claim 2, wherein the pro-polypeptide is applied in native form or in denatured form to the immobilized metal ion affinity chromatography material.

32. The method according to claim 30, wherein the immobilized metal ion affinity chromatography material is an immobilized zinc affinity chromatography material.

33. The method according to claim 31, wherein the immobilized metal ion affinity chromatography material is an immobilized zinc affinity chromatography material.

34. The method according to claim 1, wherein the protease is selected from IgA protease, trypsin, granzyme B; or combinations thereof and wherein the solution free of denaturing agents comprises 0.5 to 1.5 M Tris at a pH value of about pH 8.

35. The method according to claim 2, wherein the protease is selected from IgA protease, trypsin, granzyme B; or combinations thereof and wherein the solution free of denaturing agents comprises 0.5 to 1.5 M Tris at a pH value of about pH 8.

36. The method according to claim 1, wherein the polypeptide is a non-glycosylated polypeptide.

37. The method according to claim 2, wherein the polypeptide is a non-glycosylated polypeptide.

38. The method according to claim 3, wherein the polypeptide is a non-glycosylated polypeptide.

39. The method according to claim 16, wherein the polypeptide is a non-glycosylated polypeptide.

40. The method according to claim 1, wherein the polypeptide is apolipoprotein A-I or a fusion polypeptide comprising apolipoprotein A-I or human insulin-like growth factor 1 (IGF-1) or a fusion polypeptide comprising insulin-like growth factor i (IGF-1).

41. The method according to claim 2, wherein the polypeptide is apolipoprotein A-I or a fusion polypeptide comprising apolipoprotein A-I or human insulin-like growth factor 1 (IGF-1) or a fusion polypeptide comprising insulin-like growth factor i (IGF-1).


SEQUENCE LISTING (21 sequences)

SEQ ID NO: 1 - protein, 309 aa, Artificial Sequence
  interferon and tag and cleavage site containing tetranectin-apolipoprotein A-I fusion polypeptide
CDLPQTHSLGSHHHHHHGSVVAPPAPIVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 302 aa, Artificial Sequence
  short leader and tag and cleavage site containing tetranectin-apolipoprotein A-I fusion polypeptide
MRGSHHHHHHGSVVAPPAPIVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 3 - protein, 298 aa, Artificial Sequence
  tag and cleavage site containing tetranectin- apolipoprotein A-I fusion polypeptide
HHHHHHGSVVAPPAPIVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 4 - protein, 283 aa, Artificial Sequence
  tetranectin-apolipoprotein A-I fusion polypeptide
IVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 5 - protein, 5 aa, Artificial Sequence
  protease cleavage site
PAPSP

SEQ ID NO: 6 - protein, 4 aa, Artificial Sequence
  protease cleavage site
PPSP

SEQ ID NO: 7 - protein, 4 aa, Artificial Sequence
  protease cleavage site
PPAP

SEQ ID NO: 8 - protein, 4 aa, Artificial Sequence
  protease cleavage site
PPTP

SEQ ID NO: 9 - protein, 4 aa, Artificial Sequence
  protease cleavage site
PPGP

SEQ ID NO: 10 - protein, 6 aa, Artificial Sequence
  enzymatic cleavage site
PRPPTP

SEQ ID NO: 11 - protein, 7 aa, Artificial Sequence
  enzymatic cleavage site
VVAPPAP

SEQ ID NO: 12 - protein, 7 aa, Artificial Sequence
  enzymatic cleavage site
VVAPPSP

SEQ ID NO: 13 - protein, 7 aa, Artificial Sequence
  enzymatic cleavage site
VVAPPTP

SEQ ID NO: 14 - protein, 7 aa, Artificial Sequence
  enzymatic cleavage site
VVAPPGP

SEQ ID NO: 15 - protein, 6 aa, Artificial Sequence
  enzymatic cleavage site
APPAAP

SEQ ID NO: 16 - protein, 6 aa, Artificial Sequence
  enzymatic cleavage site
PRPPAP

SEQ ID NO: 17 - protein, 6 aa, Artificial Sequence
  enzymatic cleavage site
PRPPSP

SEQ ID NO: 18 - protein, 6 aa, Artificial Sequence
  enzymatic cleavage site
PRPPGP

SEQ ID NO: 19 - protein, 9 aa, Homo sapiens
CDLPQTHSL

SEQ ID NO: 20 - protein, 6 aa, Artificial Sequence
  hexahistidine affinity tag
HHHHHH

SEQ ID NO: 21 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK